(19) United States
(12) Patent Application Publication
(10) Pub. No.: US 2010/0248318 A1
(43) Pub. Date: Sep. 30, 2010

(54) HYDROGEN PROCESSING AND IMPURITY REMOVAL AND CLEANING METHODS IN A BIOMASS CONVERSION PROCESS

(75) Inventors: Cesar B. Granda, College Station, TX (US); Mark T. Holtzapple, College Station, TX (US); Richard R. Davison, Bryan, TX (US)

Correspondence Address: CONLEY ROSE, P.C.;David A. Rose, P. O. BOX 3267, HOUSTON, TX, 77253-3267, US

(73) Assignee: TEXAS A&M UNIVERSITY SYSTEMS, College Station, TX (US)

(21) Appl. No.: 12/795,377

(22) Filed: Jun. 7, 2010

Related U.S. Application Data
(62) Division of application No. 11/948,506, filed on Nov. 30, 2007.
(60) Provisional application No. 60/868,251, filed on Dec. 1, 2006.

(51) Int. Cl. C12P 7/62; C12P 7/54

(52) U.S. Cl. 435/135; 435/140

(57) ABSTRACT

In one embodiment, the disclosure includes a method of biomass conversion including fermenting biomass to produce a carboxylic acid or carboxylate salt and hydrogen gas, recovering the hydrogen gas, and converting the carboxylic acid or carboxylate salt to an alcohol using the hydrogen gas. In one embodiment, the hydrogen produced by biomass conversion may be converted to an acetate. Another embodiment relates to a biomass conversion system. The system may include: a fermentation unit for fermentation of biomass to a carboxylic acid or carboxylate salt in a fermentation broth and for production of a carbon dioxide and hydrogen gas stream, an extraction unit for extracting the carboxylic acid or carboxylate salt from the fermentation broth, a gas extraction unit for separation of the hydrogen gas and the carbon dioxide, and a production unit for production of an alcohol from the carboxylic acid or carboxylate salt using the hydrogen gas.

### CROSS-REFERENCE TO RELATED APPLICATIONS

This application is a divisional application which claims the benefit under 35 U.S.C. §121 of U.S. patent application Ser. No. 11/948,506, filed Nov. 30, 2007, which claims priority under 35 U.S.C. §119(e) to U.S. Provisional Patent Application No. 60/868,251, filed Dec. 1, 2006, the content of each of which is hereby incorporated herein by reference in its entirety.

### STATEMENT REGARDING FEDERALLY SPONSORED RESEARCH OR DEVELOPMENT

Not Applicable.

### BACKGROUND

1. Field of the Invention

The present invention, in some embodiments relates to a biomass conversion process.

2. Background of the Invention

MixAlco Fermentations of biomass initially produce carboxylic acids, which are then esterified. These esters then undergo a costly hydrogenation process to form mixed alcohols that can be used as fuels; thus if hydrogen were produced as a free gas by a fermentation, it would greatly lower the costs of the overall process.

### SUMMARY

In one embodiment, the invention relates to a method of biomass conversion. The method may include fermenting biomass to produce a carboxylic acid or carboxylate salt and hydrogen gas, recovering the hydrogen gas, and converting the carboxylic acid or carboxylate salt to an alcohol using the hydrogen gas.

In a specific embodiment, the hydrogen gas is recovered from a stream of carbon dioxide and hydrogen gas. Recovering may include one or a combination of several processes including: extraction of carbon dioxide from the stream using an amine absorption unit, absorption of carbon dioxide from the stream using ash, purifying hydrogen gas from the stream using a membrane, purifying hydrogen gas from the stream using a pressure swing adsorption, purifying hydrogen gas from the stream using compression following by chilling or cooling, which may also produce liquid carbon dioxide, and purifying hydrogen gas from the stream using a membrane.

In additional embodiments, the carboxylic acid or carboxylate salt may be converted to a primary alcohol or a secondary alcohol. It may pass through a ketone stage in the process.

In other embodiments, various buffers may be used in the fermentation including NH₄HCO₃ or CaCO₃.

In some embodiments, the carboxylic acid or carboxylate salt may be extracted using a high molecular weight amine, which may then further have its impurities be removed using a solid or a liquid and then be recycled to the extraction step.

In other embodiments, the carboxylic acid or carboxylate salt may be converted to an alcohol using a high molecular weight alkyl ester, which may then further have its impurities be removed using a solid or a liquid and then be recycled to the extraction step.

In one embodiment, the hydrogen produced by biomass conversion may be converted to an acetate. This may be recycled into the overall process, for example it may be added to the fermentation step.

Finally, one embodiment of the invention relates to a biomass conversion system. The system may include: a fermentation unit for fermentation of biomass to a carboxylic acid or carboxylate salt in a fermentation broth and for production of a carbon dioxide and hydrogen gas stream, an extraction unit for extracting the carboxylic acid or carboxylate salt from the fermentation broth, a gas extraction unit for separation of the hydrogen gas and the carbon dioxide, and a production unit for production of an alcohol from the carboxylic acid or carboxylate salt using the hydrogen gas.

There are many advantages to the current invention, some advantages which certain embodiments may have include:
Production of hydrogen in mixed-culture anaerobic fermentation from biomass under buffered conditions for use with other fermentation products.
Methods for purification of hydrogen from carbon dioxide.
Integration of purification methods with and within downstream processing allows efficient utilization of hydrogen from anaerobic fermentation and gasification for the production of biofuels (i.e., primary and secondary alcohols).
Integration of impurity removal and cleaning in the downstream process.
Hydrogen is an important reactant in the process for producing mixed alcohol fuels from biomass; however, it is somewhat expensive and difficult to obtain. Being able to produce it in the fermentation and integrating its purification with and within the downstream steps of the system improves the convenience and economics of the process.
Being able to produce hydrogen in the fermentation and its integration with and within the downstream process give more flexibility in the products that can be made.
The impurity removal and cleaning process is efficient in avoiding accumulation of impurities in the system. The processing shown in one embodiment gives flexibility in how efficient one desires to be in avoiding material losses, which may depend on the economics.

### BRIEF DESCRIPTION OF THE DRAWINGS

The following figures form part of the present specification and are included to further demonstrate certain aspects of the present invention. The invention may be better understood by reference to one or more of these drawings in combination with the description of embodiments presented herein.

For a more detailed description of embodiments of the invention, reference will now be made to the accompanying drawings, wherein:

FIG. 1 illustrates System A, a system for converting biomass to carboxylic acids using NH₄HCO₃ buffer, according to an embodiment of the present invention;

FIG. 2 illustrates System B, a system for converting biomass to carboxylic acids using NH₄HCO₃ buffer, according to an embodiment of the present invention;

FIG. 3 illustrates System C, a system for converting biomass to carboxylic acids using CaCO₃ buffer, according to an embodiment of the present invention;

FIG. 4 illustrates System D, a system for converting biomass to ketones and secondary alcohols using CaCO₃ buffer, according to an embodiment of the present invention;

FIG. 5 illustrates a variant of System A, a system for converting biomass to primary alcohols using NH₄HCO₃ buffer, according to an embodiment of the present invention;

FIG. 6 illustrates a variant of System C, a system for converting biomass to primary alcohols using CaCO₃ buffer, according to an embodiment of the present invention.

FIG. 7 illustrates the use of acetogenic fermentation to convert the hydrogen produced in fermentation to acetate, according to an embodiment of the present invention;

FIG. 8 illustrates Process A, an amine absorption unit for extraction of carbon dioxide from a hydrogen/carbon dioxide stream, according to an embodiment of the present invention;

FIG. 9 illustrates Process B, the use of ash for the absorption of carbon dioxide from a hydrogen/carbon dioxide stream, according to an embodiment of the present invention;

FIG. 10 illustrates Process C, the use of a membrane for purification of hydrogen from a hydrogen/carbon dioxide stream, according to an embodiment of the present invention;

FIG. 11 illustrates Process D, the use of Pressure Swing Adsorption (PSA) for the purification of hydrogen from a hydrogen/carbon dioxide stream, according to an embodiment of the present invention;

FIG. 12 illustrates Process E, the use of compression followed by chilling/cooling for the purification of hydrogen from a hydrogen/carbon dioxide stream and production of liquid carbon dioxide, according to an embodiment of the present invention;

FIG. 13 illustrates Option A and Option B for the conversion of carboxylic acids into secondary or primary alcohols, according to an embodiment of the present invention;

FIG. 14 illustrates Box A, a method of solid impurity removal and cleaning of a high molecular weight amine, according to an embodiment of the present invention;

FIG. 15 illustrates Box B, a method of liquid impurity removal and cleaning of a high molecular weight amine, according to an embodiment of the present invention;

FIG. 16 illustrates Box C, a method of solid impurity removal and cleaning of high molecular weight alkyl esters, according to an embodiment of the present invention;

FIG. 17 illustrates Box D, a method of liquid impurity removal and cleaning of high molecular weight alkyl esters, according to an embodiment of the present invention;

FIG. 18 illustrates a titration used in determining the amount of hydrogen gas produced by fermentation, according to an embodiment of the present invention; and

FIG. 19 illustrates the later steps of a MixAlco Process, according to an embodiment of the present invention.

### DETAILED DESCRIPTION

This invention includes methods for processing hydrogen gas (i.e., purification and utilization for the production of alcohols) produced in anaerobic fermentations and from gasification of the undigested solids from said fermentation. The anaerobic fermentation mainly converts biomass to carboxylic acids using a mixed culture of microorganisms, but it also produces fermentation gas which contains carbon dioxide and hydrogen gas. Buffers (e.g., calcium carbonate, ammonium bicarbonate) are used to neutralize the produced acids; therefore, the final product from the fermentation is carboxylate salts. These carboxylate salts may be dewatered and processed into alcohols, for example they may be esterified then hydrogenated. Hydrogenation is normally expensive, but may be performed at lower costs using hydrogen gas produced by fermentation.

Further, in this biomass conversion process, certain streams contain impurities that must be removed; therefore, methods for removing impurities and cleaning these streams, where impurities are likely to accumulate, are also included in this invention.

Therefore, this experiment was designed to determine if hydrogen gas is present and if so, in what concentration is hydrogen produced in the gases of an anaerobic fermentation of paper fines and dried chicken manure, in a water mixture. The inoculum was used to grow microorganisms to carry out the fermentation and ammonium bicarbonate was the buffer. An increase in the total useful energy produced by the fermentation process is possible because hydrogen may be extracted and used later in the MixAlco Process to form mixed alcohols out of the esters obtained from the carboxylic acids made in the fermentation mixture.

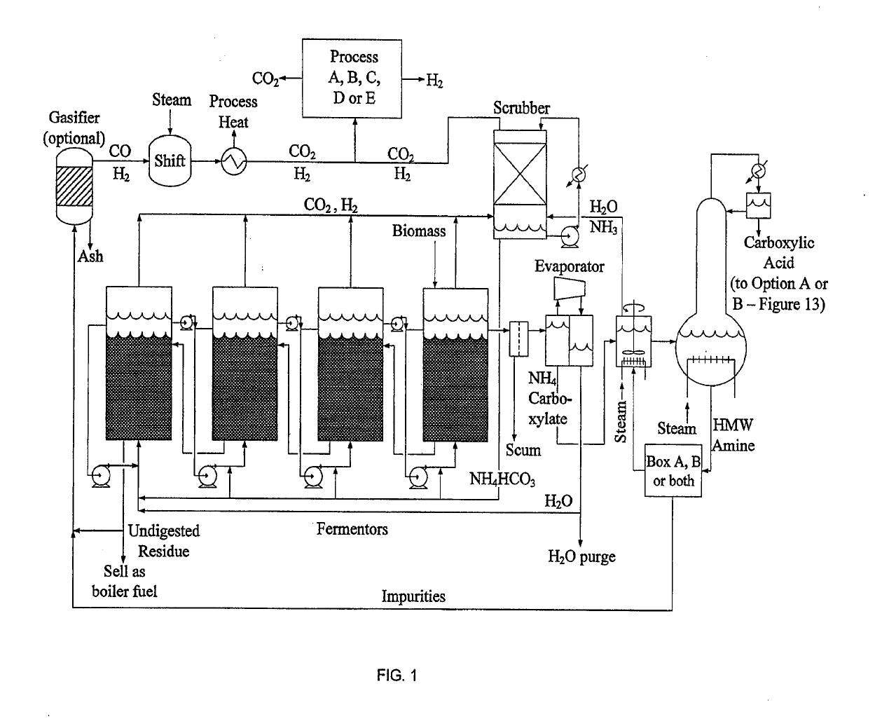

The upstream stages of the processes shown in FIGS. 1 through 7 show a method for producing carboxylate salts from biomass. Many fermentor geometry arrangements have been described previously and may be used in these upstream stages of the process. Here, the process employs four countercurrent fermentors as an example. The solids in these fermentors are added to the top and removed from the bottom. Fresh biomass is added to the right-most fermentor. Undigested residues are removed from the bottom and sent to the adjacent fermentor. This process is repeated until undigested residues are removed from the left-most fermentor. If desired, a screw press or other suitable dewatering device can be employed to reduce the liquid content in the solids that are transferred from one fermentor to the other.

Fresh water is added to the left-most fermentor. A portion of the fermentor liquid is sent to the adjacent fermentor. This process is repeated until fermentation broth is harvested from the right-most fermentor. Each fermentor is equipped with a circulation loop that allows good distribution of methane inhibitor (e.g., iodoform, bromoform, bromoethane sulfonic acid) and buffer (ammonium bicarbonate or calcium carbonate). The buffer reacts with carboxylic acids produced from digesting biomass, thus forming carboxylate salts of ammonium or calcium according to the buffer used. A mixed-culture of acid-forming anaerobic microorganisms is employed in the fermentation. The source of microorganisms can be from a variety of habitats, such as soil or cattle rumen. In one embodiment, the best results may be obtained using an inoculum from marine environments; these organisms have adapted to high-salt environments.

The fermentor temperature is controlled by regulating the temperature of the circulating liquid. The fermentor pH is regulated by the addition rate of buffer. The optimal pH is around 7.

The undigested residue leaving the left-most fermentor is a lignin-rich product that can be sold or used as boiler fuel, but it may also be gasified (as shown in FIGS. 1 through 6) to produce synthesis gas (hydrogen and carbon monoxide). This synthesis gas can then be shifted using steam to form more hydrogen and convert the carbon monoxide into carbon dioxide. From this process, heat is produced which can be used to provide energy for the rest of the plant.

The fermentation broth harvested from the right-most fermentor may have scum present, which may often be undesirable in the downstream processing steps. The scum can be removed via a variety of methods. For example, the fermentation broth can be pumped through an ultrafiltration or microfiltration membrane with a molecular weight cut-off that allows the carboxylate salts to pass, but scum is retained. Alternatively, a coagulant or flocculant can be added (such as those employed to clarify sugar juice extracted from sugarcane), which would allow the scum to be removed by filtration. If calcium carbonate is used as the buffer, lime may be added followed by carbon dioxide addition to precipitate calcium carbonate. As calcium carbonate precipitates, it entraps scum, thus removing it. The calcium carbonate and scum is then simply removed by filtration.

The de-scummed or clarified fermentation broth contains a dilute concentration of the carboxylate salts (e.g., 1 to 10%). The water is removed to form a nearly saturated solution (35 to 50%). Although a variety of dewatering methods can be employed, here a vapor compression system is shown. Vapors from the concentrated salts solution are compressed, which allows them to condense in a heat exchanger. The heat of condensation in the condenser provides the needed heat of evaporation in the boiler, thus, the heat is recycled. The process, in this example, is driven by a small amount of shaft work provided by a compressor, but other compressing devices, such as jet ejectors, may also be used.

It was found that hydrogen gas is also produced in the fermentation, and may be recovered and utilized. In the laboratory, using as fermentation substrate 80% paper and 20% chicken manure and controlling the pH with ammonium bicarbonate buffer, an average of about 6% hydrogen in the gas (carbon dioxide and hydrogen) has been measured (2% and 12% being the lowest and the highest concentrations). This amount is significant and may be recovered for use in hydrogenation processes, where it may thereby decrease the cost of alcohol production overall.

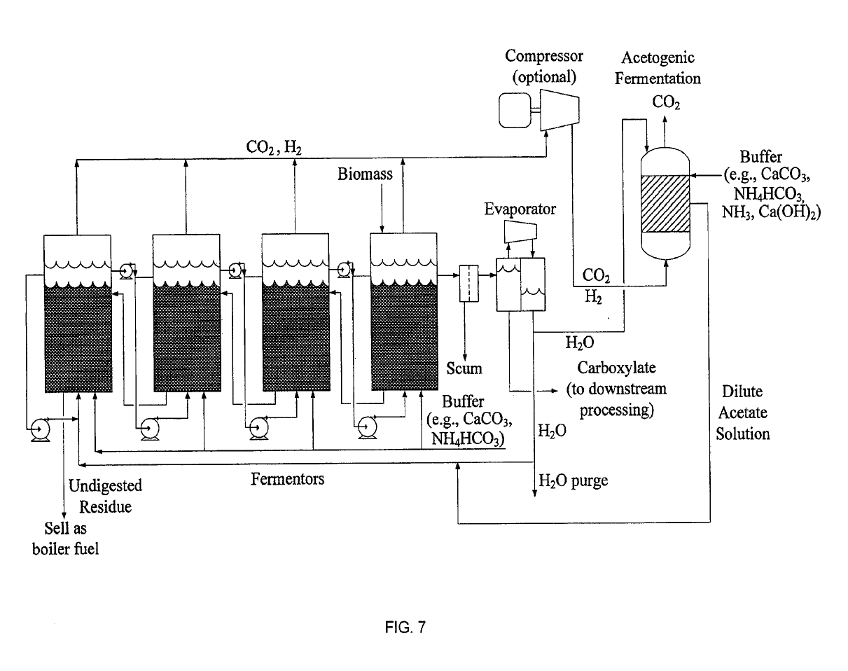

FIG. 7 shows an always-available embodiment of performing acetogenic fermentation to convert some of the carbon dioxide and all the hydrogen to acetate. A buffer (e.g., ammonium bicarbonate, ammonia, calcium carbonate, calcium hydroxide) is supplied to control the pH. From this fermentation, a dilute acetate solution is obtained which may be simply recycled to the fermentors. Operating the acetogenic fermentor at higher pressures may allow for higher acetate concentrations.

In the fermentation gas (which is mostly carbon dioxide and hydrogen), most of the carbon dioxide produced comes from the buffer (calcium carbonate or ammonium bicarbonate), which is released as the buffer neutralizes the acids formed. This carbon dioxide is known as abiotic CO₂, as opposed to the biotic CO₂, which is formed from the bacterial metabolic pathways during biomass bioconversion. In FIGS. 1 through 3 and FIGS. 5 and 6, the abiotic CO₂ is removed from the fermentation gas in an effort to recover or regenerate the buffer.

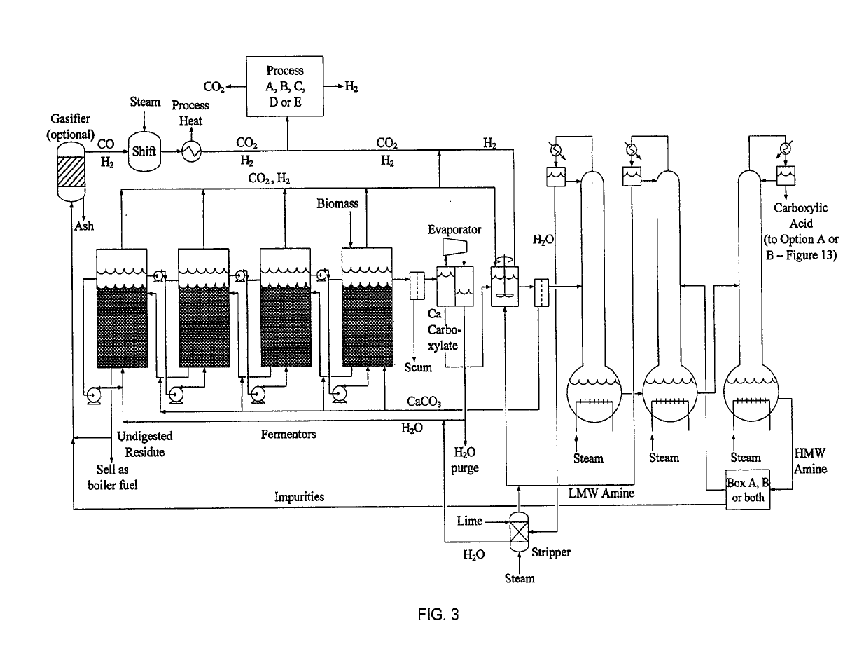

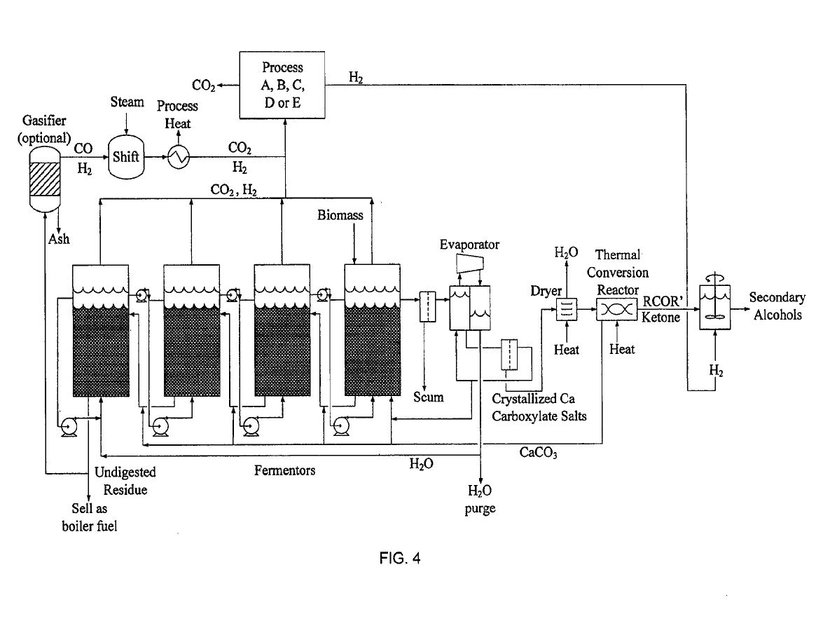

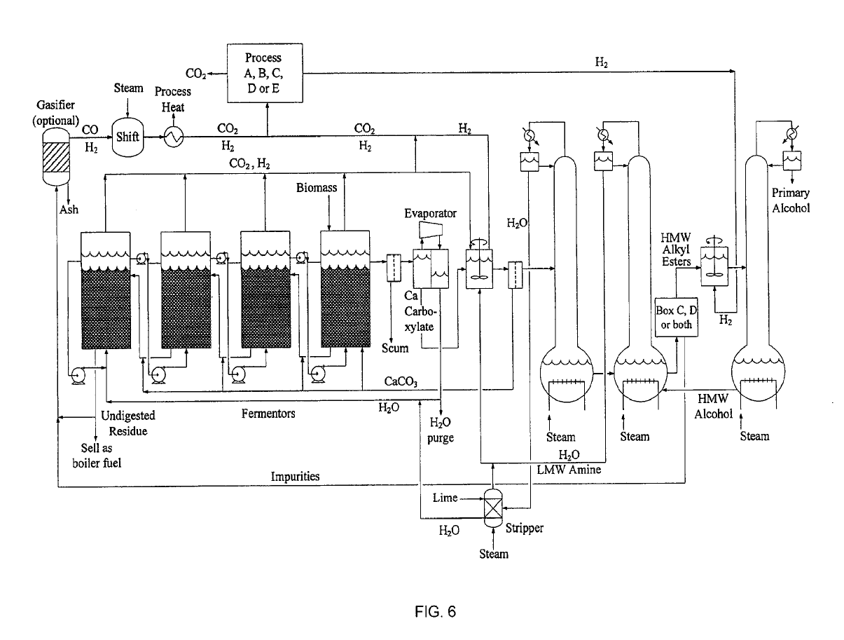

Thus, in FIGS. 1, 2 and 5, where ammonium bicarbonate is used as the buffer, the ammonia recovered downstream is contacted with the fermentation gas in a scrubber, where ammonium bicarbonate is produced and recycled back to the fermentation. Similarly, in FIGS. 3 and 6, where calcium carbonate is used as the buffer, some of the fermentation gas (i.e., the amount containing the abiotic CO₂) is sent to a reactor to allow the exchange of the calcium ions in the calcium carboxylate salt solution from the evaporator with a low-molecular-weight (LMW) tertiary amine (e.g., trimethylamine, trieuhylamine, tripropylamine, tributylamine), resulting in the formation of a LMW amine carboxylate and in the precipitation of calcium carbonate buffer, which is recycled back to the fermentation. In FIG. 4, the process shown does not require the downstream addition of CO₂; thus, the abiotic CO₂ typically cannot be removed. The resulting left-over gas from the removal of the abiotic CO₂ contains less carbon dioxide (only the biotic CO₂) and is richer in hydrogen, so economies in further hydrogen purification in this gas stream can be expected as opposed to the gas stream in FIG. 4, where the abiotic CO₂ typically cannot be removed.

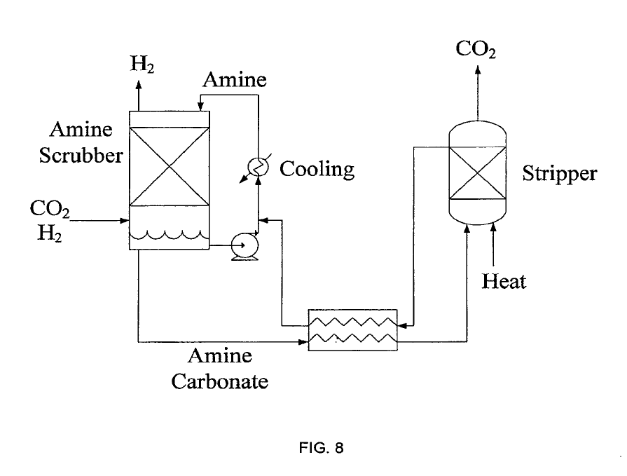

The resulting left-over gas stream after the removal of the abiotic CO₂ in FIGS. 1 through 3 and FIGS. 5 and 6, and all the fermentation gas in FIG. 4, can be treated using Processes A, B, C, D or E (depicted in FIGS. 8 through 12, respectively) or any combination of them to allow the separation of the hydrogen from the carbon dioxide. The carbon dioxide and hydrogen stream from the gasifier/shift reaction may also be sent to Processes A through E.

In FIG. 1, the concentrated ammonium carboxylate solution from the evaporator is sent to a well-mixed reactor where it is contacted with a high-molecular weight (HMW) tertiary amine (e.g., trioctylamine, triethanolamine). Because HMW amines such as trioctylamine are not very soluble in water, the reactor must be well-mixed and, if necessary, a surfactant might be added. In this well-mixed reactor, the remaining water is driven off, which causes the ammonium carboxylate salts to split, forming HMW amine carboxylate and releasing ammonia, which is sent to the scrubber to remove the abiotic CO₂ from the fermentation gas and recover the ammonium bicarbonate buffer. The resulting HMW amine carboxylate is sent to a reactive distillation column, where temperatures are increased above 200° C. At this point, the HMW amine carboxylate thermally cracks into carboxylic acids and the HMW amine (at 1 atm, typical cracking temperatures are 150 to 200° C., depending on the molecular weight of the acid). The acids leave the top of the column and the HMW amine in the reboiler is recycled back to the reactor to repeat the process.

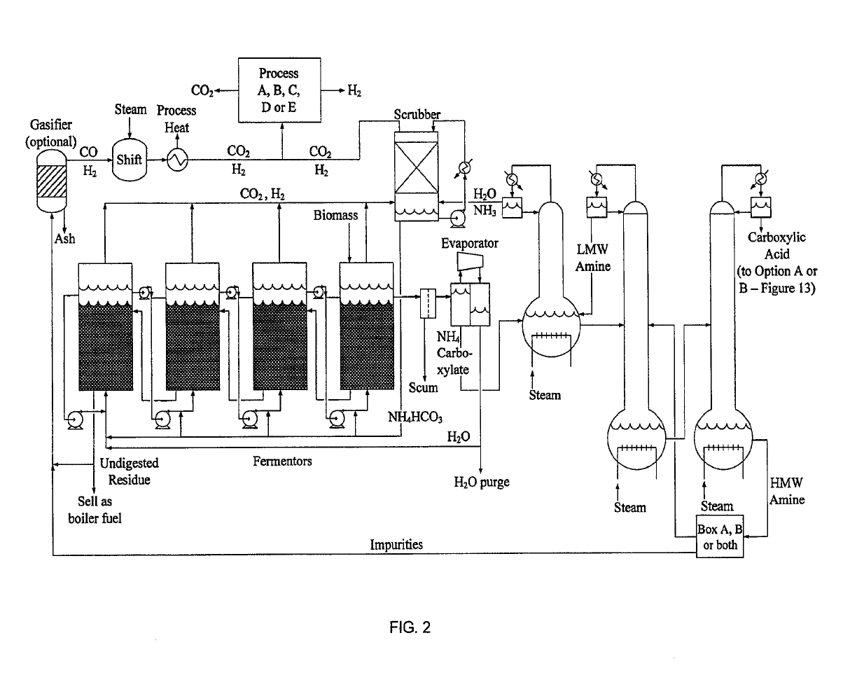

The process in FIG. 2 is similar to the process in FIG. 1 with the difference that a LMW tertiary amine (e.g., trimethylamine, triethylamine, tripropylamine, tributylamine) is used first to drive the ammonia off. Although primary and secondary amines can also be employed, tertiary amines are preferred because amide formation is avoided. The LMW amine is more soluble in water than HMW amines such as trioctylamine, which could make the process more efficient. The concentrated ammonium carboxylate solution from the evaporator is sent to a distillation column where it contacts the LMW amine. In this column, all the ammonia and most (or all) of the water are driven off. Please note that in this case trimethylamine and triethylamine are not recommended because they are more volatile than water, unless some means of recovering them from the water/ammonia stream are implemented. Alternatively, only the ammonia may be driven off first in a separate column or reactor, allowing for the LMW amine to react and form the LMW amine carboxylate. Then, in another distillation column, the water and any unreacted LMW amine are separated from the LMW amine carboxylate. The unreacted LMW amine can be steam stripped from the water before the water is sent to fermentation. If this alternative process is chosen, then trimethylamine and triethylamine may be used. Following this, the LMW amine carboxylate is then contacted in another column with a HMW amine (e.g., trioctylamine), which causes the amines to switch. The LMW amine is driven off through the top of the column and recycled back to the process leaving a HMW amine carboxylate. Then, in the same way as in FIG. 1, the HMW amine carboxylate is thermally cracked in yet another column to produce the carboxylic acids, which leave at the top, while the HMW amine in the reboiler is recycled back to repeat the process.

In FIG. 3, the process also produces carboxylic acids, but it deals with calcium carboxylate salts rather than ammonium carboxylate salts, which are produced by using calcium carbonate as the buffer rather than ammonium carbonate or bicarbonate. The concentrated calcium carboxylate solution from the evaporator is contacted in a reactor with a LMW amine (e.g., trimethylamine, triethylamine, tripropylamine, tributylamine) and carbon dioxide from the fermentation gas is added. Calcium carbonate precipitates from this reaction and is recycled to the fermentation, and a LMW amine carboxylate is formed. The LMW amine carboxylate solution, which still contains some water, is sent to a distillation column where most (or all) of the water is separated, leaving at the top of the column. Any unreacted LMW amine still present in the water is steam stripped before sending the water back to fermentation. Lime is added to the stripper to ensure that the LMW amine is not in ionic form. The LMW amine carboxylate is then sent to a second column where it is switched with a HMW amine, forming a HMW amine carboxylate, while the LMW amine leaves at the top and is recycled. As in FIGS. 1 and 2, in a third column, the HMW amine carboxylate is thermally cracked into the carboxylic acid and the HMW amine, which is recycled to repeat the process.

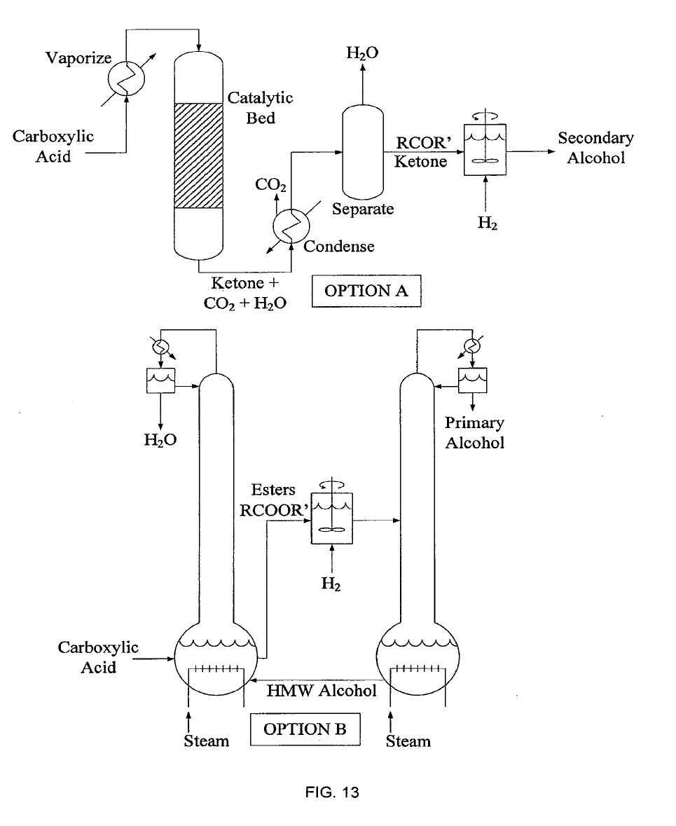

In FIGS. 1 through 3 carboxylic acids are produced. These acids can be further processed into alcohols using Options A or B depicted in FIG. 13. In Option A, the carboxylic acids are vaporized and then sent through a catalytic bed where a catalyst (e.g., zirconium oxide) is used to convert the acids into ketones, water, and carbon dioxide. After separating the carbon dioxide and the water, the ketones can then be hydrogenated with the hydrogen from the fermentation and/or gasification purified using one or a combination of the Processes A through E (FIGS. 8 though 12) in FIGS. 1 through 3. A catalyst (e.g., Raney nickel, platinum) may be employed in this hydrogenation. The final product from this hydrogenation is secondary alcohols. Alternatively, in Option B, the carboxylic acids can be esterified using a HMW alcohol (e.g., hexanol, heptanol, octanol). This is done in a distillation column while constantly removing water from the top. The resulting HMW alkyl esters can then be hydrogenolyzed (i.e., split by the addition of hydrogen) in a separate reactor using a catalyst (e.g., Raney nickel) with the hydrogen from the fermentation and/or gasification purified using one or a combination of the Processes A through E (FIGS. 8 through 12) in FIGS. 1 through 3. From this hydrogenolysis, the HMW alcohol and the corresponding primary alcohol from the carboxylic acids are obtained. A second distillation column is used to separate the HMW alcohol from the primary alcohol product, which leaves the column at the top, while the HMW alcohol at the bottoms is recycled back to the esterification.

FIG. 4 depicts the process where the fermentation is done using calcium carbonate as the buffer; therefore, calcium carboxylate salts are the product. These salts are concentrated using the evaporator until they precipitate or crystallize out of solution. The crystallized calcium carboxylate salts are filtered out of the mother liquor and sent to a dryer, while the mother liquor in the filtrate is recycled back to the concentrating side of the condenser. To avoid accumulation of impurities, some of the mother liquor may be bled off and sent back to the fermentation where the impurities will eventually leave the process in the undigested product. The dry crystallized calcium carboxylate salts are sent to a thermal conversion unit where they are heated to about 400° C. and converted into ketones. A by-product from this reaction is calcium carbonate, which is recycled back to the fermentation. The ketones are then hydrogenated in a reactor using a catalyst (e.g., Raney nickel) in the same way as in Option A in FIG. 13, using the hydrogen from the fermentation and/or gasification purified using one or a combination of the Processes A through E (FIGS. 8 through 12) in FIG. 4. The final product from this process is secondary alcohols.

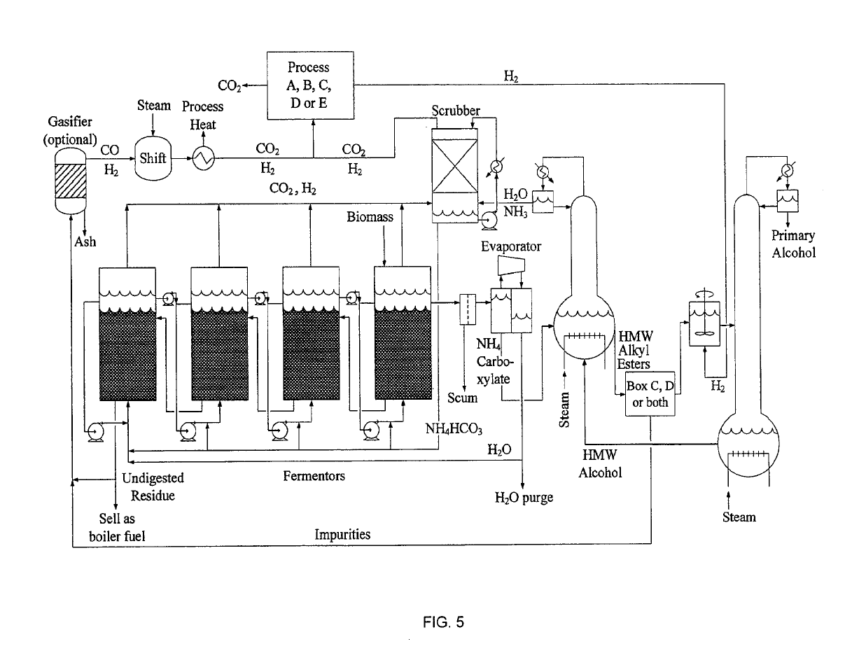

FIG. 5 illustrates a process for the direct production of primary alcohols from ammonium carboxylate salts without producing carboxylic acids first as in Option B in FIG. 13. The concentrated ammonium carboxylate solution from the evaporator is sent to an esterification column where it is contacted with a HMW alcohol (e.g., hexanol, heptanol, octanol) to be esterified in a manner similar to the carboxylic acids FIG. 13, Option B). As the esterification takes place, water and ammonia are continuously removed from the top of the column to drive the equilibrium towards the HMW alkyl esters. The resulting HMW alkyl esters are then hydrogenolyzed in a reactor using the hydrogen from the fermentation and/or gasification after purification with one or a combination of the Processes A through E (FIGS. 8 through 12) as shown in FIG. 5. From this hydrogenolysis, the corresponding primary alcohols are produced and the HMW alcohol is recovered. A second distillation column is used to separate the primary alcohol product, which exits at the top, and the HMW alcohol, which exits at the bottoms and is recycled back to the esterification.

The process in FIG. 6 is similar to FIG. 5, with the difference that, because it is processing calcium carboxylate salts, the calcium ions must be switched with a LMW amine first before performing the esterification. This switching is done in the same manner as in FIG. 3. The concentrated calcium carboxylate solution from the evaporator enters a reactor and it is contacted with carbon dioxide from the fermentation gas and a LMW amine (e.g., trimethylamine, triethylamine, tripropylamine, tributylamine). This causes calcium carbonate to precipitate, which is recycled to the fermentation, and produces a LMW amine carboxylate. The LMW amine carboxylate is sent to another distillation column where most (or all) of the water is removed through the top. Any unreacted LMW amine is steam stripped from this water stream before sending it to the fermentation. The LMW amine carboxylate is then sent to an esterification column where it is contacted with a HMW alcohol (e.g., hexanol, heptanol, octanol) to produce HMW alkyl esters. The water of reaction and LMW amine are continuously removed from the top of the column, while the HMW alkyl esters leave at the bottom. The esters are then sent to a reactor where they are hydrogenolyzed with the purified hydrogen (Processes A, B, C, D or E in FIGS. 8 through 12) from the fermentation gas and/or the gasification as shown in FIG. 6. From this hydrogenolysis, the corresponding primary alcohols and the HMW alcohol are obtained. In a third column the primary alcohol product, which leaves at the top, is separated from the HMW alcohols which leaves at the bottoms and is recycled back to the esterification.

FIG. 8 shows Process A, which is a typical amine system for the removal of carbon dioxide. Hydrogen and carbon dioxide enter the system and are contacted by an amine. The amine absorbs the carbon dioxide forming an amine carbonate. Pure hydrogen then leaves this amine scrubber. The amine carbonate is then sent to a stripper where it is heated splitting the carbon dioxide from the amine. Carbon dioxide leaves the system and the amine is then recycled to repeat the process.

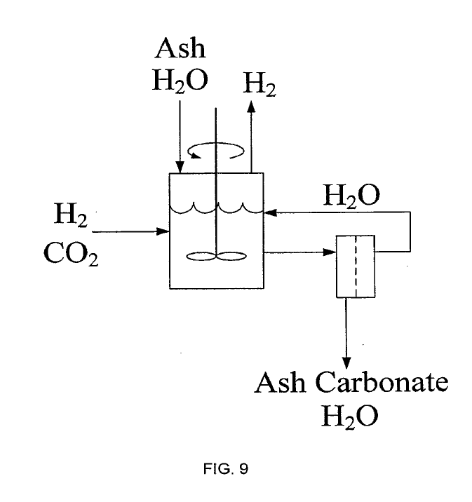

FIG. 9 shows Process B. In this process, the hydrogen/carbon dioxide stream contacts ash (from the boiler or from the gasifier) in water. Ash, being alkaline, absorbs the carbon dioxide, thus purifying the hydrogen. The resulting carbonate ash may then be returned to the fields and used as fertilizer.

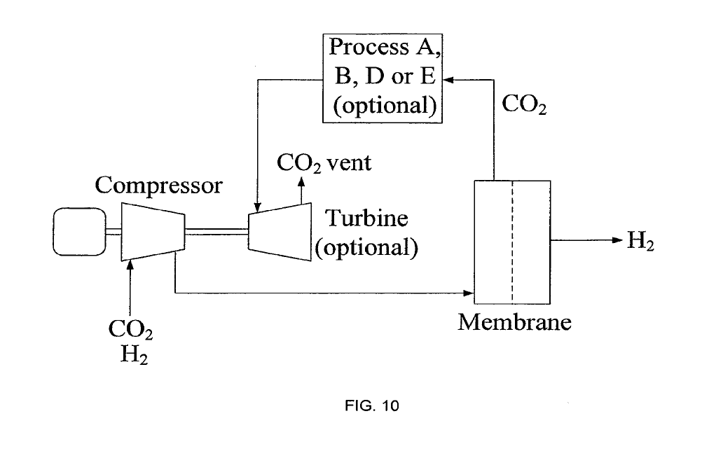

FIG. 10 shows Process C. In this process, the hydrogen/carbon dioxide stream is pressurized and sent to a membrane (e.g., palladium membrane), which is permeable to hydrogen but not to carbon dioxide. The hydrogen in the permeate is pure. The reject or retentate stream still has some hydrogen, so it may be sent to Process A, B, D or E for further hydrogen recovery. As an option, the carbon dioxide stream, which still is at a high pressure, may be sent to a turbine from which some work may be recovered before venting.

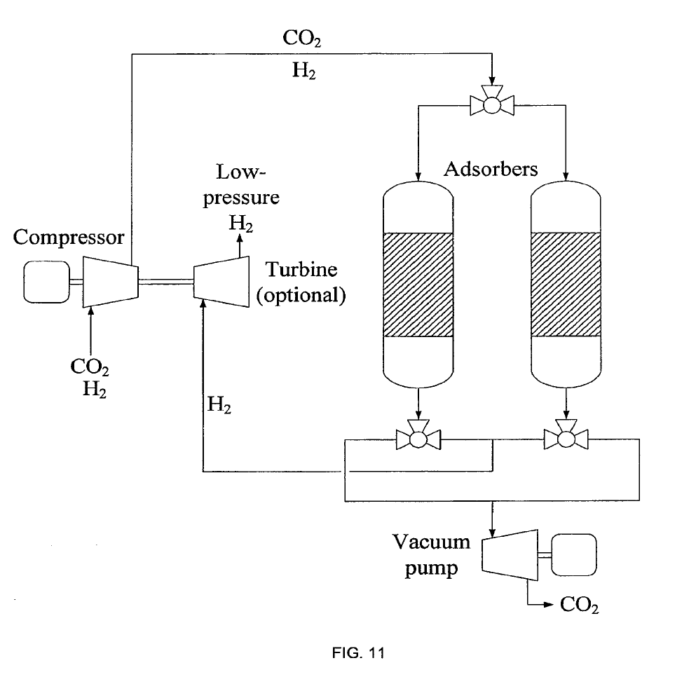

FIG. 11 illustrates Process D, which is a typical Pressure Swing Adsorption (PSA) system. In PSA, two or more adsorbers are used to adsorb impurities or unwanted components from gas streams for purification. In FIG. 11 only two adsorbers are shown as an example but more can be added. In FIG. 11, the hydrogen/carbon dioxide stream is pressurized and sent through one adsorber, but not the other. A three-way valve ensures that only one adsorber is doing the adsorption at any given time. Carbon dioxide is adsorbed and pure hydrogen leaves the system. At the same time, the other adsorber is being desorbed by applying a vacuum. Pure carbon dioxide leaves the system through the vacuum pump. Again, three-way valves keep the vacuum pump from suctioning the adsorbing side at any given time. Once the adsorbing side becomes saturated with carbon dioxide, the three-way valves are switched and then the vacuum is applied to this adsorber to start desorption, while the other adsorber starts receiving the pressurized hydrogen/carbon dioxide stream to commence the adsorbing mode. This switching from one adsorber to the other allows for virtually continuous processing of the gas stream.

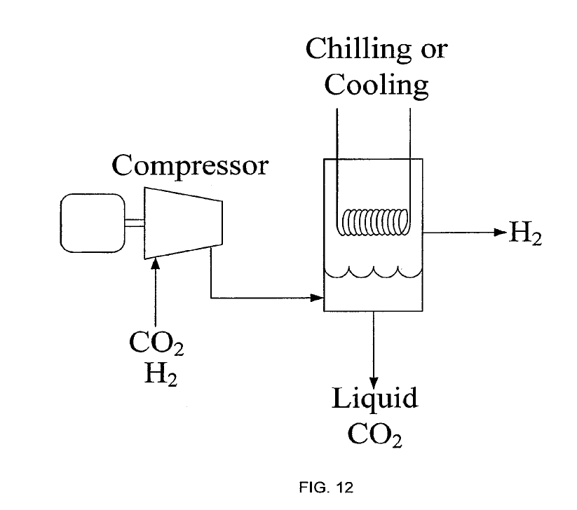

FIG. 12 shows Process E, which consists of pressurizing the hydrogen/carbon dioxide stream and applying either chilling or cooling depending on the pressure. The product from this process, besides the pure hydrogen, is liquid carbon dioxide, which can be sold into the chemical or food markets.

In FIGS. 1 through 3 and 5 through 6, impurity removal may be necessary in streams such as the HMW-amine stream and the HMW-alkyl-ester stream. Box A or B or both A and B in series and Box C or D or both C and D in series may be used as shown in those figures.

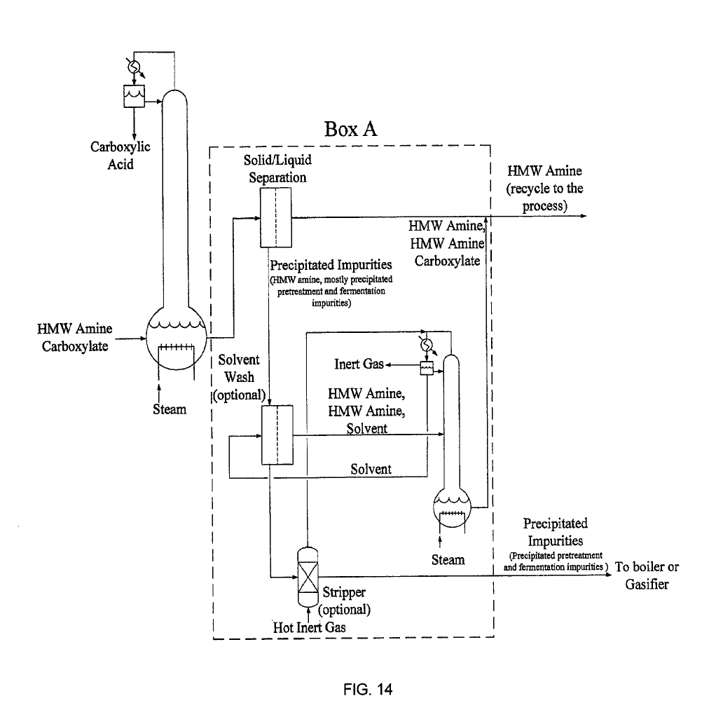

FIG. 14 shows Box A, a process for impurity removal and cleaning of the HMW-amine stream in the production of carboxylic acids as shown in FIGS. 1 through 3. This particular process depicted in FIG. 14 has been disclosed. This method handles solid or precipitated impurities. The HMW amine goes through a solid/liquid separator (e.g., filter, centrifuge, settling tank+filter), where the solid or precipitated impurities are removed from the liquid stream. Because the solid impurities are soaked in the HMW amine, a solvent (e.g., hexane, LMW amine) may be used to wash off the HMW amine. Then the solvent/HMW amine stream is separated by distillation. The HMW amine is then recycled to the process, whereas the solvent is recycled to repeat the washing. Optionally, hot or warm inert gas (e.g., N₂, Ar) can be blown through the solids to strip any remaining solvent and sent to the distillation condenser to recover it. In this condenser/accumulator, the inert gas is dislodged from the solvent and recycled. The solid impurities are then sent to the gasifier or boiler for combustion.

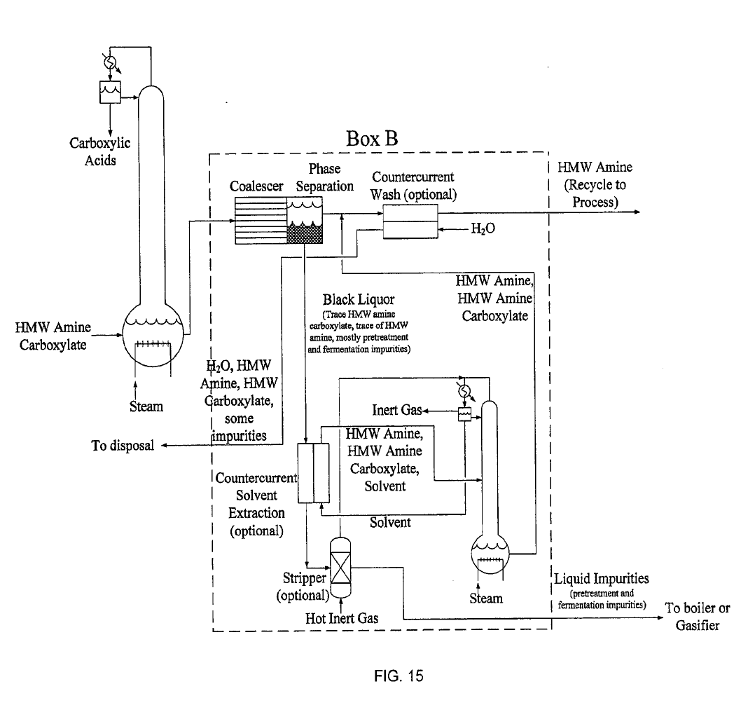

FIG. 15 illustrates Box B, which also removes impurities and cleans the HMW amine stream (e.g., trioctylamine) in carboxylic acid production as depicted in FIGS. 1 through 3. This method handles liquid non-precipitable impurities, which are water soluble and scarcely soluble in the HMW amine. The HMW amine goes to a coalescer, where the HMW-amine phase and the impurity phase are allowed to separate. The impurities are decanted and thus separated. As an option, the HWM-amine phase can be countercurrently washed with water to further purify it. The water from this wash is simply disposed of. However, such option is not recommended as some impurities in the HMW-amine stream are tolerable and the washing will cause some losses of the HMW amine to this waste water stream unless countercurrent extraction with a solvent (e.g., hexane) is used for its recovery. The impurity phase can, if desired, undergo a countercurrent solvent (e.g., hexane, LMW amine) extraction to recover any HMW amine (or HMW amine carboxylate) lost in this stream. Then, the HMW amine/solvent stream can be separated by distillation to recycle the HMW amine and the solvent to their corresponding part of the process. After exiting the countercurrent solvent extraction, the impurities are saturated with solvent, which can be recovered, if desired, by steam stripping it with hot inert gas (e.g., N₂). Then, this stream from the stripper is sent to the distillation condenser, where the solvent is condensed and recovered and the inert gas dislodges from the liquid to be recycled. The solvent-free impurities are then sent to the boiler or gasifier for combustion.

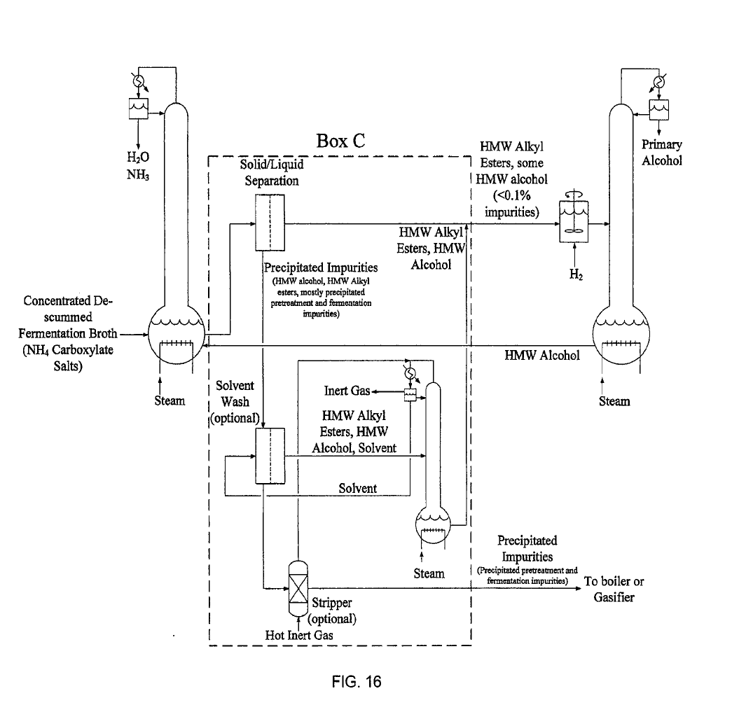

FIG. 16 shows Box C, which removes impurities and purifies the HMW-alkyl ester stream before it is sent to hydrogenolysis. In general, hydrogenation and hydrogenolysis catalysts are susceptible to poisoning and the presence of impurities may cause undesired hydrogen consumption; therefore, it may be necessary to attain high purities in this ester stream. Box C, just as Box A, handles solid or precipitated impurities. The HMW alkyl esters leaving the esterification column are sent to a solid/liquid separator (e.g., filter, centrifuge, settling tank+filter), where the solids are segregated from the liquid. The liquid leaving the separator will likely contain mostly the HMW alkyl esters, some HMW alcohol and <0.1% impurities. The solid impurities, which are soaked in the HMW alkyl esters, can be washed using a solvent (e.g., hexane). The solvent and the HMW alkyl esters are then separated by distillation to recycle the solvent back to the extraction and to send the HMW alkyl esters to distillation. As an option, the solid impurities can be stripped of the solvent by blowing through them hot inert gas (e.g., N₂, CO₂). This stream is then sent to the distillation condenser, where the solvent condenses and it is recovered and the inert gas is dislodged from the liquid and recycled. Finally, the impurity stream may be sent to the boiler or gasifier for combustion.

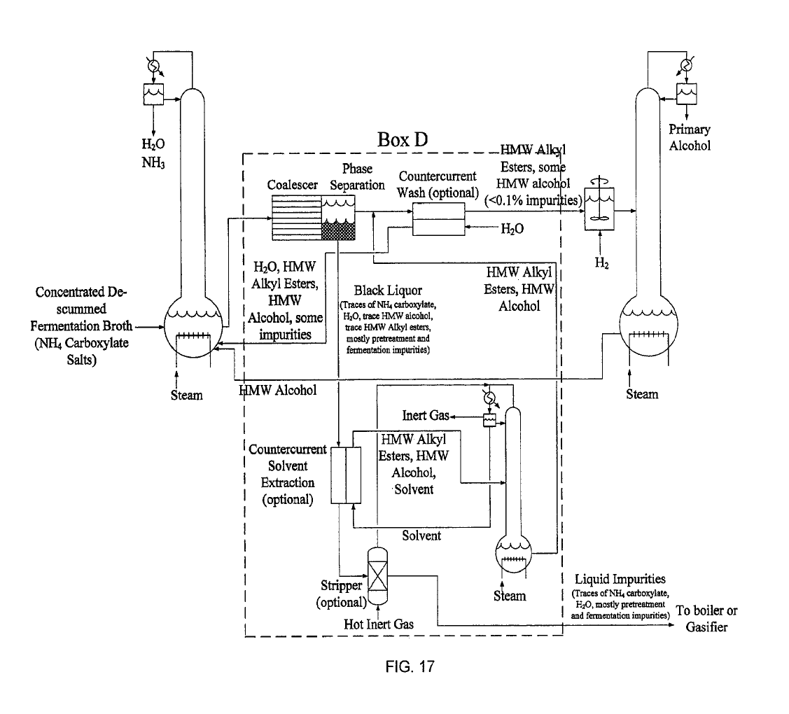

FIG. 17 depicts Box D for removing impurities and purifying the HMW-alkyl ester stream before sending it to be hydrogenolyzed. Just as Box B, Box D handles liquid non-precipitable impurities, which are soluble in water but scarcely miscible in the HMW alkyl-ester phase. The HMW-alkyl-ester stream exiting the esterification column is sent to a coalescer, where the phases are allowed to separate. The impurity phase is decanted, and the HMW-alkylester phase is sent to a countercurrent wash, which is necessary to give it its final polish for the high purities that might be needed for hydrogenolysis. The wash water from the countercurrent wash, which is saturated with the HMW alkyl esters, may be sent back to the esterification, if desired, so that losses may be avoided. The decanted impurities from the coalescer, which are saturated with the HMW alkyl esters (and some HMW alcohol), have the option to undergo countercurrent solvent extraction to recover the esters (and the alcohol) that might otherwise be lost. The solvent (e.g., hexane) and the HMW alkyl esters are then separated by distillation, recycling the solvent and sending the esters to hydrogenolysis. If recovery of the solvent from the solvent saturated impurity stream from the countercurrent extraction is desired, hot inert gas (e.g., N₂, CO₂) can be used to strip the solvent off the impurities. The inert gas/solvent stream is sent to the distillation condenser, where the solvent is condensed and recovered and the inert gas dislodges from the liquid and is recycled. Lastly, the solvent-free impurities are sent to the boiler or gasifier.

The choice of any of the optional processing in FIGS. 14 through 17 may be dictated by cost-considerations.

### EXAMPLES

The following examples are included to demonstrate specific embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

Fermentation Make Up. The fermentation mixture contained an 80% paper and 20% manure ratio with a final make up of 16 grams of paper fines, 4 grams of manure, 225 mL of water mixture (H₂O, Na₂S, Cysteine, HCl), and 25 mL of seed inoculums, the source of the microorganisms, six 1-L reaction flasks, one reactor with exactly half of all the components in a 500-mL flask, and two reactors with exactly 3/20 the amount of the initial components in a 150-mL reaction bottle, all fitted with a septum top. The reactants were mixed together and then nitrogen purged for 5 minutes before being sealed and continuously agitated in an incubator with a temperature near 27° C. Minimum air exposure was allowed whenever the reactor was opened (for example, to fix a broken needle) by way of nitrogen purge. Samples were set up every 2 days for 17 days so that gas concentrations could be collected and analyzed at different times during the fermentation (Domke, 2004). The objective was to determine the H₂ to CO₂ ratio produced in the fermentation gas.

The reactors were analyzed on Day 18 revealing that the ratio of hydrogen to carbon dioxide ranges from 0.01 to 0.13 mol H₂/mol CO₂ with an average of 0.07 mol H₂/mol CO₂. These results show that the hydrogen in the fermentation gas may be used as a source of hydrogen needed to hydrogenate the esters formed from the MixAlco Process to produce mixed alcohols such as seen in FIG. 19.

At the conclusion of the experiment, all nine fermentations were analyzed to determine the hydrogen to carbon dioxide ratio. The results are shown in Table 1.

TABLE 1
Hydrogen Production by Fermentation

| Sample ID | Day 1 | Day 3 | Day 5 |
|---|---|---|---|
| Component | (mol/mol %) | (mol/mol %) | (mol/mol %) |
| Hydrogen | 0.68 | 0.23 | 0.47 |
| CO₂ | 6.93 | 11.66 | 10.80 |
| O₂/Argon | 0.14 | 0.13 | 0.15 |
| Nitrogen | 90.18 | 85.73 | 86.44 |
| Total | 97.93 | 97.75 | 97.86 |
| H₂/CO₂ | 0.0981241 | 0.01972556 | 0.04351852 |
| % Hydrogen | 8.94 | 1.93 | 4.17 |
| (based on H₂ and | | | |
| CO₂ only) | | | |
| Sample ID | Day 7 | Day 9 | Day 11 |
| Component | (mol/mol %) | (mol/mol %) | (mol/mol %) |
| Hydrogen | 0.79 | 1.53 | 0.31 |
| CO₂ | 8.24 | 13.56 | 11.52 |
| O₂/Argon | 0.13 | 0.12 | 0.13 |
| Nitrogen | 88.51 | 82.90 | 85.79 |
| Total | 97.67 | 98.11 | 97.75 |
| H₂/CO₂ | 0.095873786 | 0.11283186 | 0.02690972 |
| % Hydrogen | 8.75 | 10.14 | 2.62 |
| (based on H₂ and | | | |
| CO₂ only) | | | |
| Sample ID | Day 13* | Day 15 | Day 17 |
| Component | (mol/mol %) | (mol/mol %) | (mol/mol %) |
| Hydrogen | 0.18 | 1.42 | 0.21 |
| CO₂ | 4.42 | 10.63 | 18.35 |
| O₂/Argon | 0.17 | 0.17 | 0.37 |
| Nitrogen | 92.66 | 86.07 | 79.41 |
| Total | 97.43 | 98.29 | 98.34 |
| H₂/CO₂ | 0.04072398 | 0.1335842 | 0.01144414 |
| % Hydrogen | 3.91 | 11.78 | 1.13 |
| (based on H₂ and | | | |
| CO₂ only) | | | |

*On Day 13 the reactor was not glass, but plastic, meaning the data from that day might be underestimated because plastic is more permeable to hydrogen and the plastic lid does not seal as well as the septum stoppers in the glass containers.

The tests showed the ratio of hydrogen to carbon dioxide ranged from 0.01 up to 0.13, with an average of 0.07. The lowest percent hydrogen observed was 1.93% on Day 3 and the highest was 11.78% on Day 15. The average percent hydrogen for all days tested was 5.93%.

These data prove that the reaction produces hydrogen as a by-product during the fermentation. Thus the total energy able to be recovered from the fermentation is higher than previously thought. This will greatly reduce costs in the MixAlco process because hydrogen might not need to be produced from other sources.

The reactors that maintained a pH of 6.5 for an extended period of time did not produce the largest ratio of hydrogen to carbon dioxide. As the hydrogen was produced, some of it either disappeared or reacted again. Also the hydrogen content did not seem to follow a pattern with time and instead seem to be random. This could result from hydrogen escaping from the reactors causing the ratio to drop significantly. Controlling the amount of gas that escapes may be significant in obtaining a high H₂/CO₂ ratio.

Nitrogen was present in larger amounts than H₂ and CO₂. This is expected due to the nitrogen purge. This also explains why the oxygen content in the reactor is so low; the nitrogen purge is designed to replace the oxygen with inert nitrogen gas. Thus calculations need not be performed based on the nitrogen numbers; the H₂ and CO₂ ratio is likely much more significant in this experiment.

Interspecies hydrogen transfer may have also occurred in this experiment. This allows hydrogen in the free gas phase to react with the low molecular weight carboxylic acids to form high molecular weight carboxylic acids plus carbon dioxide. If this reaction occurred, the hydrogen content of the gas was reduced.

Finally, heat may influence the H₂ and CO₂, ratio and may be controlled in some systems.

Overall, this experiment shows that hydrogen is produced by this particular fermentation mixture and could be used if it were to be separated from the rest of the gases.

### Example 2

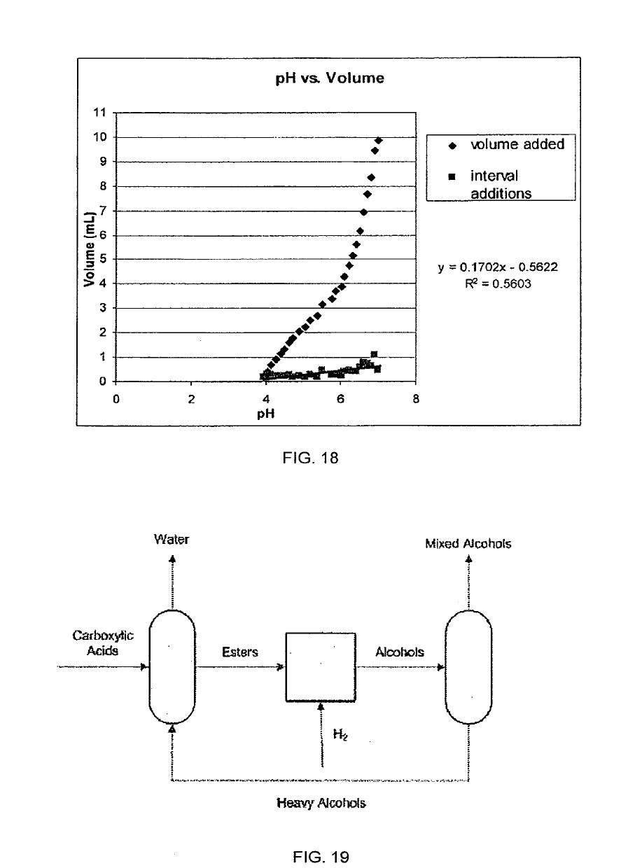

pH Maintenance. One main problem faced in batch anaerobic fermentations is maintaining the pH near neutrality with anaerobic conditions so that the microorganisms can survive and perform the fermentation. To accomplish this task, the fermentation containers were fitted with septum stoppers and 22-gauge needles attached to a three-way valve and a syringe were used to draw and test each sample. The pH was tested with pH paper ranging from 5.0 to 10.0 in 0.5 increments. If the pH was too low, a predetermined amount of 0.016 M ammonium bicarbonate solution was added to the fermentation to bring the pH back to seven. The amount added was determined based on titrations performed using diluted glacial acetic acid and the same ammonium bicarbonate solution seen in Table 2 and FIG. 18. The pH was tested as specific amounts of the ammonium bicarbonate solution were added until the pH returned to 7.0. FIG. 18 provided an approximate amount of ammonium bicarbonate solution needed to return the fermentation to neutrality because the fermentation produced primarily acetic acid. The amounts added to each reactor were altered after the first additions according to the chart and little change was observed. When the pH did not seem to drop substantially, more ammonium bicarbonate solution was added to insure that the pH did not drop to a dangerously acidic level and affect the microorganisms.

TABLE 2
pH Calibration of Samples*

| pH | Burett Volume | Volume Added | Volume from Last Interval |
|---|---|---|---|
| 3.94 | 20 | N/A | N/A |
| 4.05 | 20.19 | 0.19 | 0.19 |
| 4.15 | 20.4 | 0.4 | 0.21 |
| 4.29 | 20.68 | 0.68 | 0.28 |
| 4.39 | 20.9 | 0.9 | 0.22 |
| 4.5 | 21.13 | 1.13 | 0.23 |
| 4.62 | 21.34 | 1.34 | 0.21 |
| 4.74 | 21.6 | 1.6 | 0.26 |
| 4.88 | 21.8 | 1.8 | 0.2 |
| 5.06 | 22.04 | 2.04 | 0.24 |
| 5.19 | 22.23 | 2.23 | 0.19 |
| 5.39 | 22.5 | 2.5 | 0.27 |
| 5.5 | 22.69 | 2.69 | 0.19 |
| 5.76 | 23.13 | 3.13 | 0.44 |
| 5.87 | 23.4 | 3.4 | 0.27 |
| 6.02 | 23.68 | 3.68 | 0.28 |
| 6.1 | 23.9 | 3.9 | 0.22 |
| 6.22 | 24.31 | 4.31 | 0.41 |
| 6.33 | 24.75 | 4.75 | 0.44 |
| 6.42 | 25.18 | 5.18 | 0.43 |
| 6.5 | 25.6 | 5.6 | 0.42 |
| 6.6 | 26.18 | 6.18 | 0.58 |
| 6.71 | 26.95 | 6.95 | 0.77 |
| 6.81 | 27.69 | 7.69 | 0.74 |
| 6.9 | 28.35 | 8.35 | 0.66 |
| 7.01 | 29.44 | 9.44 | 1.09 |
| 7.05 | 29.88 | 9.88 | 0.44 |

*Calibration reading of pH meter in 7.00; standard = 7.04 pH ammonium bicarbonate solution = 8.14; 0.016 M (ammonium bicarbonate solution).

During the experiment, the fermentation samples began to decrease the pH within the first two days; however, many of the reactors did not initialize until eight days, which dropped the pH to 7.0 or below. Most reactors would fluctuate between pH 7.0 and 8.0 and then decrease very rapidly around Day 5 to pH 6.5. This seems to show that fermentation finally stabilized and acids began to be produced. The reaction appeared to occur at a very fast pace, producing enough acids to keep the pH at 6.5 regardless of the ammonium bicarbonate added daily.

### Example 3

Venting. Another concern addressed during this experiment was that hydrogen is an extremely small molecule and the container used during the fermentation was not proven to be hydrogen tight. Therefore, a thick septum stopper and a crimp seal were used to best seal the opening of the container and a 22-gauge needle was used to attach the containers to the venting line. A 25-gauge needle was initially used, but ended up being too short to allow samples to be taken, leading to the use of the 22-gauge needles. Another problem with the needles was that they would leave piercing holes in the septum, which made the septum appear flimsy; this led to the thought that they could possibly leak hydrogen gas. Therefore, once the needles had been removed on the second to last day of the experiment to allow pressure build up, all the septa were replaced so that the largest amount of hydrogen could be contained. During this procedure, the Day 13 reactor was cracked making it unusable; the fermentation was then transferred into a plastic reactor bottle and sealed using a large rubber stopper with a glass septum tube inserted in it. This reactor continuously maintained a low pH and it still resulted in a low hydrogen production that could be explained by the use of a plastic reactor bottle not being sealed as well as the other glass containers, or the fact that too much ammonium bicarbonate solution was inadvertently added to the container on Day 16.

The reactors were also attached to a venting line on the three-way valve to allow the reactors to vent into a hose that led to the hood so the reactor pressure did not build up causing the glass to crack. This ventilation and sampling technique allowed the fermentation to be exposed to a minimal amount of air during the experiment as the reactors were never left open. Once the fermentation was started, the flasks were only opened if a needle broke off in the septum requiring it to be replaced. When a septum was replaced, nitrogen purge was used to prevent any oxygen and impurities from being introduced into the reactor thus maintaining the initial conditions. On the day the final reactor was set up, the reactors were not vented over night to allow the gas pressure to build up so a large gas sample could be obtained. The septa on all the reactors were replaced and allowed to purge prior to sealing on the final day to obtain the best seal possible for the container (Aiello-Mazzarri et al., Bioresource Technology, 97:47-56 2006, incorporated by reference herein).

Although only exemplary embodiments of the invention are specifically described above, it will be appreciated that modifications and variations of these examples are possible without departing from the spirit and intended scope of the invention.

1-18. (canceled)

19. A method of biomass conversion comprising: fermenting biomass to produce a carboxylic acid or carboxylate salt and hydrogen gas; recovering the hydrogen gas; and converting the hydrogen gas to acetate.

20. (canceled)

21. The method of claim 19 wherein converting the hydrogen gas to acetate further comprises performing acetogenic fermentation.

22. The method of claim 21 wherein recovering the hydrogen gas further comprises recovering a gas stream comprising hydrogen and carbon dioxide, and wherein performing acetogenic fermentation further comprises fermenting at least a portion of the gas stream to produce acetate.

23. The method of claim 21 wherein performing acetogenic fermentation further comprises utilizing a buffer to control the pH of the acetogenic fermentation.

24. The method of claim 23 wherein the buffer is selected from the group consisting of ammonium bicarbonate, ammonia, calcium carbonate, calcium hydroxide, and combinations thereof.

25. The method of claim 21 wherein performing acetogenic fermentation further comprises operating an acetogenic fermentor at a pressure suitable to provide a desired acetate concentration.

26. The method of claim 19 further comprising recycling at least a portion of the acetate to the biomass fermentation.

27. The method of claim 19 further comprising converting the carboxylic acid or carboxylate salt to a primary alcohol.

28. The method of claim 19 further comprising converting the carboxylic acid or carboxylate salt to a secondary alcohol.

29. The method of claim 19 wherein fermenting biomass comprises using a NH₄HCO₃ buffer.

30. The method of claim 29 wherein recovering the hydrogen gas further comprises recovering a gas stream comprising hydrogen and carbon dioxide and wherein the method further comprises increasing the concentration of the hydrogen in and reducing the volume of the gas stream by utilizing carbon dioxide in the gas stream to recover ammonium bicarbonate buffer.

31. The method of claim 19 wherein fermenting biomass comprises using a CaCO₃ buffer.

32. The method of claim 31 wherein recovering the hydrogen gas further comprises recovering a gas stream comprising hydrogen and carbon dioxide and wherein the method further comprises increasing the concentration of the hydrogen in and reducing the volume of the gas stream by utilizing carbon dioxide in the gas stream to recover calcium carbonate buffer.

33. The method of claim 19 further comprising extracting the carboxylic acid or carboxylate salt using a high molecular weight amine.

34. The method of claim 33 further comprising:
removing impurities from the high molecular weight amine after the extraction step; and
recycling the high molecular weight amine to the extraction step.

35. The method of claim 34 wherein removing impurities from the high molecular weight amine after the extraction step is performed using a liquid.

36. The method of claim 19 further comprising converting the carboxylic acid or carboxylate salt to a high molecular weight alkyl ester by reaction of the carboxylic acid or carboxylate salt with a high molecular weight alcohol.

37. The method of claim 36 further comprising:
removing impurities from the alkyl ester after the conversion step; and
recycling the high molecular weight alkyl ester to the conversion step.

38. The method of claim 37 wherein removing impurities from the alkyl ester after the conversion step is performed using a liquid.

39. The method of claim 19 wherein converting the hydrogen gas to acetate further comprising recovering a gas stream comprising carbon dioxide.
